(19) United States
(12) Patent Application Publication
(10) Pub. No.: US 2023/0002477 A1
(43) Pub. Date: Jan. 5, 2023

(54) METHOD FOR TREATING INFECTION OF SEVERE ACUTE RESPIRATORY SYNDROME CORONAVIRUS 2 (SARS-COV-2)

(71) Applicant: Jiu-Yao WANG, Tainan (TW)

(72) Inventor: Jiu-Yao WANG, Tainan (TW)

(21) Appl. No.: 17/745,171

(22) Filed: May 16, 2022

Related U.S. Application Data
(60) Provisional application No. 63/218,049, filed on Jul. 2, 2021.

(51) Int. Cl. C07K 14/785; C07K 16/10; A61P 31/14

(52) U.S. Cl. CPC C07K 14/785; C07K 16/10; A61P 31/14; A61K 38/00

(57) ABSTRACT

The disclosure provides a method for treating an infection of SARS-CoV-2 infection in a subject, comprising administering to said subject a therapeutically effective amount of a peptide, which is a recombinant fragment human surfactant protein D. The disclosure also provides a pharmaceutical composition for treating an infection of SARS-CoV-2 infection comprising a therapeutically effective amount of the recombinant fragment human surfactant protein D and a pharmaceutically acceptable carrier.

### CROSS REFERENCE TO RELATED APPLICATIONS

This non-provisional application claims the benefit under 35 U.S.C. § 119(e) to U.S. Provisional Application No. 63/218,049, filed on Jul. 2, 2021, all of which is hereby expressly incorporated by reference into the present application.

### FIELD OF THE INVENTION

The present invention is related to a recombinant fragment of human surfactant protein D (rfhSP-D), which can be developed for treatment of an infection of severe acute respiratory syndrome coronavirus 2 (SARS-COV-2).

### BACKGROUND OF THE INVENTION

COVID-19 is an infectious disease caused by severe acute respiratory syndrome coronavirus 2 (SARS-CoV-2), which leads to mild to severe respiratory syndromes with an average case fatality rate of 2%. Comparing with other types of coronaviruses, SARS-CoV-2 shares 76-95% protein similarity with SARS-CoV and 29-46% protein similarity with MERSCoV. Despite the striking similarities between SARS-CoV-2 and SARS-CoV, the latter has a 10% case fatality rate.

SARS coronavirus 2 (SARS-CoV-2) is an enveloped coronavirus, belonging to the Coronaviridae family of viruses, and is genetically close to SARS-CoV (˜80% sequence similarity) and bat coronavirus RaTG13 (96.2%). The envelope of SARS-CoV-2 is coated by the spike (S) glycoprotein, a small envelope (E) glycoprotein, membrane (M) glycoprotein, nucleocapsid (N) protein, and several putative accessory proteins. The SARS-CoV-2 mediates its entry into the host cell using the 51 sub-unit of the S glycoprotein by binding to angiotensin-converting enzyme 2 (ACE2) receptor. However, viral entry into the host cells requires not only binding to the ACE2 receptor, but also priming of the S protein by a transmembrane protease serine 2 (TMPRSS2) via cleavage of the S protein at S1/S2 sites. This cleavage is very crucial for the virus-host cell membrane fusion and cell entry. Following viral replication, assembly, and release, the infected host cells undergo pyroptosis, thus, releasing Damage- Associated Molecular Patterns (DAMPs). DAMPs are then recognised by surrounding macrophages and monocytes that respond to viral infection by inducing cytokine storm. However, in some cases, an impaired or dysregulated immune response can also occur, causing an Acute Respiratory Distress Syndrome (ARDS).

Due to the global COVID-19 pandemic, it is important to develop, there is an urgent need for development of a new therapy strategy for SARS-CoV-2 infection.

### BRIEF SUMMARY OF THE INVENTION

It was unexpectedly found in the invention that a recombinant fragment of human surfactant protein D (called as “rfhSP-D” hereinafter) can interfere with the binding of SARS-CoV-2 S1 and the angiotensin-converting enzyme 2 (ACE-2) receptor. In addition, the peptide rfhSP-D was confirmed to be able to inhibit an infection of SARS-CoV-2 by the test using pseudotyped lentiviral particles expressing SARS-CoV-2 51 protein. Accordingly, the present invention provides a new approach for treating an infection of SARS-CoV-2.

In one aspect, the invention provides a method for treating an infection of SARS-CoV-2 in a subject, comprising administering to said subject a therapeutically effective amount of a recombinant fragment of human SP-D (rfhSP-D).

In one particular embodiment, the peptide rfhSP-D has amino acid sequence as set forth in SEQ ID NO: 3.

According to one certain example of the invention, the peptide rfhSP-D consists of the sequence as set forth in SEQ ID NO: 3.

According to certain embodiments of the invention, the peptide rfhSP-D is administered in an amount effective to inhibit the entry of SARS-CoV-2 into a host cell in said subject.

In another aspect, the invention provides a pharmaceutical composition for treating an infection of SARS-CoV-2 comprising a therapeutically effective amount of the rfhSP-D peptide, and a pharmaceutically acceptable carrier.

In one particular embodiment of the invention, the peptide rfhSP-D has amino acid sequence as set forth in SEQ ID NO: 3.

According to a certain example of the inventionthe peptide rfhSP-D consists of the sequence as set forth in SEQ ID NO: 3.

According to certain embodiments of the invention, the therapeutically effective amount is an amount effective to inhibit the entry of SARS-CoV-2 into a host cell in a subject.

In a further aspect, the invention provides use of a peptide rfhSP-D in the manufacture of a medicament for treating an infection of SARS-CoV-2.

According to certain embodiments of the invention, the medicament comprises a therapeutically effective amount of the peptide rfhSP-D, and a pharmaceutically acceptable carrier. In some embodiments, the therapeutically effective amount is an amount effective to inhibit the entry of SARS-CoV-2 into a host cell in a subject.

In one particular embodiment of the invention, the peptide rfhSP-D comprises the sequence as set forth in SEQ ID NO: 3.

In one certain example of the invention, the peptide rfhSP-D consists of the amino acid sequence as set forth in SEQ ID NO: 3.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 provides that the recombinant fragment of human SP-D (rfhSP-D) and recombinant human full-length SP-D (hFL-SP-D) binding with the spike (S1) (A) and its RBD (B) of the SARS-CoV-2 was determined via direct ELISA. Microtiter wells were coated with SARS-CoV-2 spike S1 protein (5 μg/ml ) (HEK 293 cells) or RBD (5 μg/ml ) (HEK 293 cells) in carbonate-bicarbonate buffer, pH 9.6 overnight at 4° C. The following day, the wells were blocked with Tris Buffered Saline (TBS) buffer containing 1% BSA and 5 mM CaCl₂, pH 7.2-7.4. After washing the wells with TBS, the wells were incubated with a series of two-fold dilutions of rfhSP-D or hFL-SP-D protein in blocking buffer at 4° C. overnight. The binding between S1 protein and rfhSP-D was detected using biotinylated mouse anti-Human SP-D detection antibody (1:180), followed by probing with Streptavidin horseradish peroxidase (HRP)-conjugate 1:40. The data were expressed as mean of three independent experiments done in triplicates ±SEM. Significance was determined using the unpaired t test statistical analysis. The error bars show SEM. Control, maltose and EDTA groups compared to 2.5 μg or 5 μg S1 (RBD) in CaCl_{2.}

FIG. 2 provides the competitive ELISA that shows the impact of Maltose and EDTA on rfhSP-D binding to S1 (A) and its RBD (B). Polystyrene microtiter plates were coated with 2 μg/ml rfhSP-D, and incubated with SARS-CoV-2 spike S1 protein (2.5 and 5 μg/ml) (sheep-IgG tag) or RBD (His-tag) (2.5 and 5 μg/ml ). The binding was detected using anti-sheep IgG HRP antibodies (1:2000) or anti-His antibodies (1:2000). Absorbance at 450 nm were recorded by VersaMax™ ELISA Microplate Reader. Significance was determined using the unpaired t test statistical analysis. The error bars show SEM. All group compared to RBD in CaCl2 (*p<0.05; **p <0.01; ***p<0.001; ****p<0.0001).

FIG. 3 provides the expression of ACE2 receptor on HEK293T cells by immunofluorescence microscopy (A), flow cytometry (B) and western blotting (C). (A) HEK293T (0.5×105 cells) and HEK293T-ACE2 cells (0.5×105 cells) were seeded on coverslips, followed by incubation at 37° C. under standard culture conditions. After washing the cells with PBS twice, the ACE2 expression was detected in both cell lines using the ACE2 antibody [SN0754](1:250), followed by incubation for 1 h at room temperature. Following PBS washes, Goat anti-rabbit IgG (H+L) cross-adsorbed secondary antibody (1:500) was added. Following PBS washes, the coverslips were mounted in medium with DAPI on a microscopy slide and viewed under a fluorescence microscope (Olympus). (B) Flow cytometric analysis of ACE2 expression was determined by the shift in the fluorescence intensity using ACE2 antibody [N1N2], N-term (GeneTex) (1:250). The ACE2 expression was detected by CytoFLEX. (C) The ACE2 expression was examined by Western blotting using ACE2 antibody [SN0754] (GeneTex) (1:1000).

FIG. 4 shows that rfhSP-D treatment inhibited the interaction between SARS-CoV-2 S1 and ACE2 receptor on HEK293T cells. Protein complex was made by tagging SARS-CoV-2 S1 protein (5 ug/ml) with anti-His antibody (10 ug/ml ), followed by incubation with rfhSP-D (0.625, 1.25, 2.5, 5 or 10 μg/ml ) for 2 h at room temperature. This complex (S1+anti-His+rfhSP-D) was added on to HEK293T-ACE2 cells (1×10⁵ cells) at 37° C. for 2 h. The cells were collected and washed with FACS buffer twice and incubated with anti-mouse IgG PE conjugate (Genetex, GTX25881) (1:100) for 30 min and washed three times. The cells stained with 51 were detected by CytoFLEX. Significance was determined using the unpaired t test statistical analysis. All groups compared to S1. The error bars show SEM. M=mock (*p<0.05; **p<0.01; ****p<0.0001) (n=3).

FIG. 5 shows that rfhSP-D acted as an entry inhibitor of SARS-CoV-2 infection. (A) The SARS-CoV-2 pseudotyped lentiviral particle and pseudotyped lentiviral particle containing medium were determined the S1 expression by western blotting. (B) Luciferase reporter activity of rfhSP-D treated HEK293T cells (overexpressing ACE2 receptor) transduced with of SARS-CoV-2 S1 pseudotyped lentiviral particles. Significance was determined using the unpaired t test statistical analysis. All groups compared to VSV-S1. The error bars show SEM. M=medium (**p<0.01; ***p<0.001; ****p<0.0001) (n=3).

### DETAILED DESCRIPTION OF THE INVENTION

The above summary of the present invention will be further described with reference to the embodiments of the following examples. However, it should not be understood that the content of the present invention is only limited to the following embodiments, and all the inventions based on the above-mentioned contents of the present invention belong to the scope of the present invention.

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The present invention provides a method for treating an infection of SARS-CoV-2 in a subject, comprising administering to said subject a therapeutically effective amount of a recombinant fragment of human surfactant protein D (rfhSP-D). Also provided herein is a pharmaceutical composition for treating SARS-CoV-2 infection comprising a therapeutically effective amount of said peptide rfhSP-D, and a pharmaceutically acceptable carrier.

The term “surfactant protein D” or “SP-D” as used therein refers to a collagen-containing C-type lectin and a member of the collectin family, is known to be involved in pulmonary surfactant homeostasis and immunity (1). SP-D is primarily synthesized and secreted into the air space of the lungs by alveolar type II and Clara cells (2, 3). Its primary structure is organized into four regions: a cysteine rich N-terminus, a triple-helical collagen region composed of Gly-X-Y triplets repeats, an a-helical coiled neck region, and a C-terminal C-type lectin or carbohydrate recognition domain (CRD) (1). As a versatile innate immune molecule, SP-D can interact with a number of pathogens, triggering clearance mechanisms against viruses, bacteria, and fungi, as well as and apoptotic cells (4). According to the invention, the natural (wild type) of SP-D was identified to have the amino acid sequence as set forth in SEQ ID NO: 1.

As used herein, the term “a recombinant fragment of human surfactant protein D” or “a fragment of a recombinant human surfactant protein D”” refers to a fragment of a recombinant human SP-D, called as a peptide “rfhSP-D” hereinafter, which comprises the amino acid residues (aa) 199 to 375 of the natural human SP-D with a mutation of Proline (P) to Serine (S) at aa 200 (i.e., aa 2 of the peptide of SEQ ID No: 3. In one particular example, the peptide rfhSP-D consists of the amino acid sequence as set forth in SEQ ID NO: 3.

The term “subject” as used herein includes a human and/or a non-human animal, such as companion animals (e.g., dogs, cats, etc.), farm animals (e.g. cattle, sheep, pigs, horses, etc.), or experimental animals (e.g., rats, mice, guinea pigs, etc.).

The term “treat,” “treating” or “treatment” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

For use in therapy, the therapeutically effective amount of the rfhSP-D is formulated as a pharmaceutical composition for administration. Accordingly, the invention further provides a pharmaceutical composition comprising a therapeutically effective amount of the rfhSP-D, and one or more pharmaceutically acceptable carriers.

For the purpose of delivery and absorption, a therapeutically effective amount of the SP-D according to the present invention may be formulated into a pharmaceutical composition in a suitable form with a pharmaceutically acceptable carrier.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents.

The composition of the present invention can provide the effect of rapid, continued, or delayed release of the peptide rfhSP-D after administration to the patient. According to the invention, the pharmaceutical composition may be adapted for administration by any appropriate route, including but not limited to oral, rectal, nasal, topical, vaginal, or parenteral route (such as intramuscular, intravenous, subcutaneous, and intraperitoneal), transdermal, suppository, and intranasal methods.

The present invention is further illustrated by the following examples, which are provided for the purpose of demonstration rather than limitation.

### EXAMPLES

### Materials and Methods

### 1. Expression and Purification of rfhSP-D

DNA sequences coding for 8 Gly-X-Y repeats of collagen region, a-helical neck and CRD region of human SP-D (SEQ ID NO: 2) were cloned under T7 promoter and expressed in Escherichia coli BL21 (1DE3) pLysS using construct pUK-D1 (25, 26). Primary bacterial inoculum (25 ml) was grown in Luria-Bertani (LB) medium (500 ml) with 34 mg/ml chloramphenicol and 100 mg/ml ampicillin (Sigma-Aldrich) at 37° C. until an OD600 of 0.6 was reached. Following isopropyl b-D-thiogalactoside (IPTG) (0.5 mM) induction, the transformed E. coli cells were grown further for another 3 h at 37° C. on a shaker. The bacterial cells were harvested by centrifugation (5000 rpm, 4° C., 10 min), and the cell pellet was re-suspended in lysis buffer containing 50 mM Tris—HC1, pH 7.5, 200 mM NaCl, 5 mM EDTA, 0.1% v/v Triton X-100, 0.1mMphenylmethane sulfonyl fluoride (PMSF) (Sigma-Aldrich), and 50 mg lysozyme/ml (Sigma-Aldrich) at 4° C. for 1 h. The lysed cell lysate was then sonicated at 60 Hz for 30 sec with an interval of 2 min (12 cycles) using a Soniprep 150 (MSE, London, UK), followed by centrifugation (12,000 rpm, 15 min). The inclusion bodies were denatured using buffer (50 ml) containing 0.5 M Tris—HCl, 0.1 M NaCl, pH7.5 and 8 M urea for 1 h at 4° C. The soluble fraction was dialysed against the same buffer containing varied concentration of urea (4 M, 2 M, 1 M, 0 M) for 2 h each. The refolded material was then dialysed against affinity buffer (50 mM Tris—HCl, pH7.5, 100 mM NaCl, 10 mM CaCl₂) for 2 h at 4° C. The affinity buffer dialysed supernatant was then loaded on to a maltose-agarose column (5 ml) (Sigma-Aldrich); the bound rfhSP-D was eluted using elution buffer containing 50 mM Tris—HCl, 100 mM NaCl, and 10 mM EDTA. The purified rfhSP-D was run on SDS-PAGE to assess its purity. LPS was removed using Endotoxin Removal Resin (Sigma-Aldrich). LPS level was determined using QCL-1000 Limulus amebocyte lysate system (Lonza) and found to be <5 pg/mg of the rfhSP-D. The rfhSP-D was identified as having the amino acid sequence of SEQ ID NO: 3, which comprises the amino acid residues (aa) 199 to 375 of the natural human SP-D with a mutation of Proline (P) to Serine (S) at aa 200 (i.e., aa 2 of the peptide of SEQ ID No: 3).

### 2. ELISA

Polystyrene microtiter plates (Sigma-Aldrich) were coated with SARS-CoV-2 spike S1 protein (NativeAntigen S1. NCBI accession number YP_009724390.1 AA1-674, produced in HEK 293 cells; Acro, AA Val 16 -Arg 685, accession # QHD43416.1, produced in HEK 293 cells) or RBD (Acro, Arg319-Phe541, accession #QHD43416.1, produced in HEK 293 cells) (27) (5 μg/ml, 100 μl/well) at 4° C. overnight using carbonate/bicarbonate (CBC) buffer, pH 9.6 (Sigma-Aldrich). The following day, the microtiter wells were washed three times with Tris Buffered Saline-Tween (TBST, pH 7.2-7.4) containing 0.05% v/v Tween 20 (Sigma-Aldrich) and 5 mM CaCl₂ (Thermo Fisher Scientific). The wells were then blocked by TBS containing 1% w/v BSA and 5 mM CaCl_{2,} for 1 h. After washing three times with TBST, the wells were incubated with two-fold dilutions of rfhSP-D or recombinant human full-length SP-D (hFL-SP-D, R&D, 1920-SP, produced in HEK 293 cells) protein (100 ml/well) in the blocking buffer at 4° C. overnight. Next day, the wells were washed and then incubated with biotinylated mouse anti-Human SP-D detection antibody (1:180) (R&D Systems) for 2 h at room temperature. After washing, the wells were incubated with Streptavidin horseradish peroxidase (HRP)-conjugate (1:40; R&D System) for 20 min, followed by washing three times. TMB substrate (100 ml/well; Thermo Fisher Scientific) was added to each well and the reaction was stopped using 1M H₂SO₄ (50 ml/well; Sigma-Aldrich). Absorbance at 450 nm were measured by VersaMax™ ELISA Microplate Reader.

### 3. Competition ELISA

Polystyrene microtiter plates were coated with 2 μg/ml rfhSP-D (100 μ1/well) at 4° C. overnight using CBC buffer and washed three times with TBS buffer containing 0.05% v/v Tween 20 and 5mM CaCl₂. The wells were blocked with TBS containing 1% BSA and 5 mM CaCl₂ for 1 h. The wells were then washed three times and incubated with SARS-CoV-2 spike S1 protein (sheep-IgG tag) or RBD (His-tag) (2.5 or 5 μg/ml, 100 μl/well) separately in blocking buffer containing 10 mM maltose and 10 mM EDTA at 4° C. overnight. Next day, the wells were washed and then incubated with anti-sheep IgG-HRP antibodies (Genetex, GTX27111, 0.5 μg/ml, 100 μl/well) (1:2000) or anti-His antibodies (Genetex, GTX628914, 0.5 μm/ml, 100 μl/well) (1:2000) for 2 h. For the detection of RBD binding, the wells were further incubated with anti-mouse IgG antibody (Abcam, ab6728, 0.5 μg/ml, 100 μl/well) (1:2000) for 2 h. After washing, the plates were incubated with TMB substrate (100μl/well) and then quenched with 1M H₂SO₄ (50 μ1/well). Absorbance at 450 nm was recorded by VersaMax™ ELISA Microplate Reader.

### 4. Western Blotting

HEK293T and HEK293T-ACE2 cells (0.5×10⁵) were lysed by RIPA buffer (Thermo Fisher Scientific) containing protease inhibitor (AMRESCO VWR life sciences) on ice for 15 minutes and then centrifugation (13000 rpm, 4° C., 15 min). 30 μg samples resuspended in Laemmli sample buffer (10 μl) and heated at 100° C. for 10 minutes. The samples were loaded into an SDS-PAGE (8% v/v) gel and then electrophoretically transferred onto the PVDF membrane (320 mA for 2 h) (Sigma-Aldrich) in transfer buffer [25 mM Tris—HCl pH 7.5, 190 mM glycine (Sigma-Aldrich), and 20% v/v methanol (Thermo Fisher Scientific)]. The membranes were blocked by 5% w/v dried milk powder (Sigma-Aldrich) diluted in TBS+ 0.05% v/v Tween 20 (TBST) for 1 h at room temperature and incubated with anti-SARS-CoV-2 (COVID-19) Spike antibody (GeneTex, GTX135356; 1:1000) or anti-ACE2 antibody [SN0754] (GeneTex, GTX01160; 1:1000) at 4° C. overnight. The membranes were washed three times and probed with secondary Goat anti-rabbit IgG horseradish peroxidase (HRP)-conjugate (1:10000; Fisher Scientific) for 1 h at room temperature. Following TBST washes, the protein expression was measured by Western Lightning Plus ECL (PerkinElmer) and chemiluminescent detection was performed using FluorChem R system (ProteinSimple, San Jose, Calif., USA).

### 5. Cell Culture and Treatments

Human embryonic kidney (HEK) 293T or HEK293T cells overexpressing ACE2 receptor (HEK293T-ACE2) were cultured in complete Gibco Dulbecco's Modified Eagle Medium (DMEM), supplemented with 10% v/v fetal bovine serum (FBS), 100 U/ml penicillin (Sigma-Aldrich) and 100 μg/ml streptomycin (Sigma-Aldrich), and left to grow at 37° C. in the presence of 5% v/v CO2 for approximately 48 h before passaging. ACE2-stably expressing HEK-293T cells were selected by Blasticidin S HCl (Thermo Fisher Scientific). Since HEK293T cells were adherent, they were detached using 2× Trypsin-EDTA (0.5%) (Thermo Fisher Scientific) for 10 min at 37° C. Cells were then centrifuged at 1,500 rpm for 5min, followed by re-suspension in complete DMEM medium. To determine the cell count and viability, an equal volume of the cell suspension and Trypan Blue (0.4% w/v) (Thermo Fisher Scientific) solution were vortexed, followed by cell count using a hemocytometer with Neubauer rulings (Sigma-Aldrich). Cells were then re-suspended in complete DMEM for further use.

### 6. Generation of ACE2-Stably Expressing

HEK-293T Cells Human ACE2 gene was amplified fromMGC library (cDNA clone MGC:47598) by using Kapa HiFi PCR kit (Kapa Biosystems), and sub-cloned into NheI and EcoRI sites of pLAS2w.Pbsd (a lentiviral transfer vector from RNA core, Academia Sinica, Taiwan) by using GenBuilderTMCloning kit (GeneScript®). For generation of VSVG pseudotyped lentivirus carrying human ACE2 gene, three plasmids (pCMV-DR8.91, pLAS2w.ACE2.Pbsd and pMD.G) were transiently transfected into HEK-293T cells by using TransIT®-LT1 transfection reagent (Mirus). The culture medium was harvested to infect HEK-293T cells, and then the infected cells were selected with 5 mg/ml blasticidin for one week to generate HEK-293T-ACE2 stable cells.

### 7. Flow Cytometry

ACE2 expression was assessed between HEK293T cells overexpressing ACE2 receptor (HEK293T-ACE2) and HEK293T cells alone using flow cytometry. Briefly, both ACE2-transfected and non-transfected HEK293T cells (1×10⁵ cells) were incubated with ACE2 antibody [N1N2, N-term (GeneTex, GTX101395), (1:250)] for 1 h at room temperature. Following PBS washes, the cells were probed with Goat anti-Rabbit IgG (H+L) Cross-Adsorbed Secondary Antibody linked to Alexa Fluor 647 (Thermo Fisher Scientific) (0.6 μl/100 μl per tube) for 1 h at room temperature in dark. After washing with PBS, the cells were resuspended in FACS buffer (PBS containing 2% FBS) and subjected to flow cytometry.

For binding experiment using the rfhSP-D, SARS-CoV-2 S1 protein containing a C-terminal His-tag (Acro; S1N-C52H3) (5 μg/ml ) was tagged with anti-His antibody (Genetex; GT359) (1:100) at 4° C. for 1 h, followed by pre-incubation with a series of two-fold dilutions of rfhSP-D (10 μg/ml ) or mock (medium only) at 4° C. for 1 h. HEK293T-ACE2 cells (1×10⁵ cells) were incubated in DMEM incomplete medium with the mixture of SARS-CoV-2 S1 protein, anti-His antibodies and rfhSP-D at 37° C. for 2 h. The cells were collected and washed with FACS buffer twice and incubated with anti-mouse IgG-PE conjugate (Genetex, GTX25881) (1:100) for 30 min and washed three times. The live cells were gated from FSC vs. SSC dot plot in order to determine the PE positive cells containing S1 on their surface by CytoFLEX.

### 8. Fluorescence Microscopy

HEK293T and HEK293T-ACE2 cells (0.5×10⁵) were grown on coverslips in complete DMEM medium overnight under standard culture conditions, as mentioned above. Next day, cells were washed with PBS three times, the coverslips were fixed with 4% v/v paraformaldehyde (Sigma-Aldrich) for 15 minutes, and then washed twice. The coverslips were permeabilized with 0.25% v/v Triton-100 (Sigma-Aldrich) for 15 min. After washing, coverslips were blocked with 2% w/v BSA for lh and incubated with ACE2 antibody [SN0754 (1:250) (GeneTex, GTX01160)], followed by Goat anti-rabbit IgG (H+L) cross-adsorbed secondary antibody (1:500) (Thermo Fisher Scientific) for 1 h at room temperature in dark. After incubation with secondary antibody, the cells were washed twice with PBS and mounted in the medium with DAPI (Abcam) on the slides to visualize under an upright fluorescence microscope (BX51; Olympus).

### 9. Production of SARS-CoV-2 Pseudotyped Lentivirus

The pseudotyped lentivirus carrying SARS-CoV-2 spike protein was generated by transiently transfecting HEK293T cells with pCMV-DR8.91, pLAS2w.Fluc.Ppuro and pcDNA3.1-nCoVSD18 (SARS-CoV-2 spike gene with 54 nucleotides deletion at its C-terminus was synthesized and cloned into pcDNA3.1 expression vector). HEK293T cells were seeded one day before, and then transfected with the indicated plasmids using TransIT®-LT1 transfection reagent (Mirus). The culture medium was replenished at 16 h and harvested at 48 h and 72 h post-transfection. Cell debris was removed by centrifugation at 4,000 x g for 10 min, and the supernatant was passed through 0.45-mm syringe filter (Pall Corporation). The pseudotyped lentivirus was aliquoted and stored at −80° C. until further use. The transduction unit (TU) of SARS-CoV-2 pseudotyped lentivirus was estimated using cell viability assay in response to the limited dilution of lentivirus. In brief, HEK293T cells, stably expressing human ACE2, were plated on 96-well plate one day before lentivirus transduction. For titrating, different amounts of lentivirus particles were added to the culture medium containing polybrene (final concentration 8 mg/ml). Spin infection was carried out at 1,100 x gin 96-well plate for 30 min at 37° C.

After Incubating cells at 37° C. for 16 h, the culture medium containing virus particles and polybrene was removed and replaced with fresh complete DMEM containing 2.5 mg/ml puromycin. After treating with puromycin for 48 h, the culture media was removed, and the cell viability was assessed using 10% AlarmaBlue reagents, according to manufacturer's instruction. The survival of uninfected cells (without puromycin treatment) was set as 100%. The virus particle titer (TU) was determined by plotting the survival of cells versus diluted viral dose.

### 10. Pseudotyped Virus Neutralization Assay

HEK293T cells in 10 cm Petri dishes were transfected with pCMVDR8.91, pcDNA nCoV-SD18 and pLAS2w.FLuc.Ppuro plasmids (5, 2, 8 μg, respectively). Next day, cells were washed with PBS gently, and replaced with 10 ml of fresh medium (RPMI containing 10% FBS). The medium at 48 and 72 h were collected and stored in −80° C. for future use. HEK293T-ACE2 cells (HEK293T cells overexpressing ACE2 receptor) (0.5×10⁵ cells) were pre-incubated with rfhSP-D (0, 5, 10 and 20 μg/ml ) for 24 h and then washed twice with PBS. The SARS-CoV-2 pseudotyped lentiviral particle containing medium (500 μl/well) was added on to the cells, followed by incubation at 37° C. under standard culture conditions. After 2 h, fresh complete DMEM medium (500 μl) was added on to the cells and incubated at 37° C. Following 72 h incubation, the cells were washed with PBS twice, and incubated with lysis buffer at 37° C. for 10 min. Firefly luciferase activity (RLU) was measured using ONEGlo™ Luciferase Assay System (Promega) and FlexStation.

### 11.Statistical Analysis

GraphPad Prism 6.0 software was used to generate all the graphs. Unpaired t test was used for the statistical analysis. The significance values were considered between rfhSP-D treated and untreated conditions, based on *p<0.05. Error bars show the SEM (figure legends).

RESULTS

1. Interaction of rfhSP-D and Recombinant Human Full-Length SP-D (hFL-SP-D) With 51 Protein and Its RBD

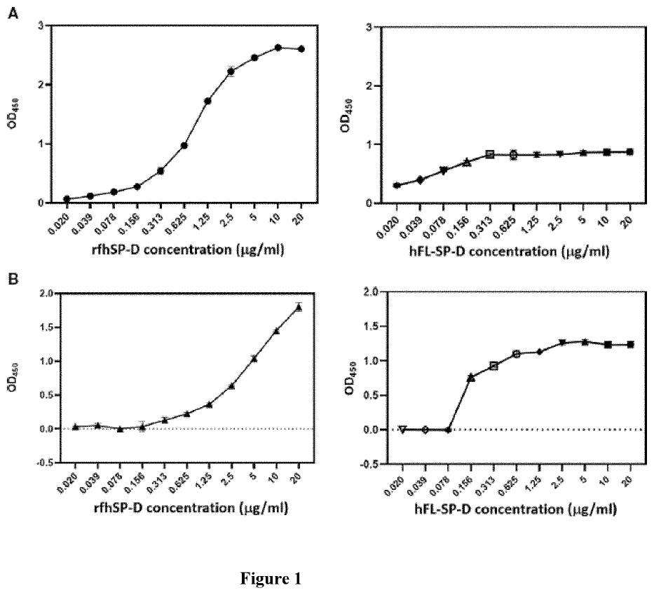

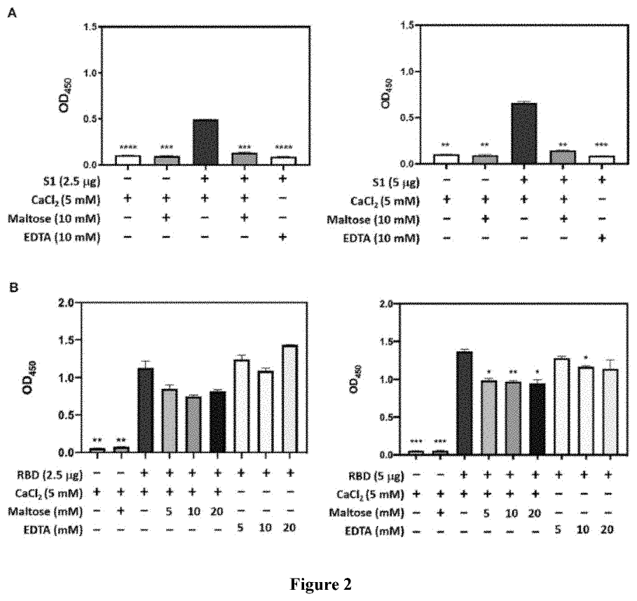

In SARS-CoV, S-protein is the predominant surface glycoprotein recognized by the host innate immune system. The S protein of SARS-CoV-2 has almost 76% identity to SARS-CoV. Previous studies indicated that SP-D bound S protein of SARS-CoV which required Ca²⁺; the binding was inhibited by maltose. Therefore, the first part of this study was aimed at examining the interaction of LPS-free rfhSP-D and hFL-SP-D with spike protein (Si) using direct binding ELISA. It was found that rfhSP-D/hFL-SP-D bound SARS-CoV-2 Si protein in a dose-dependent manner (FIG. 1A); this interaction was inhibited by maltose and EDTA (FIG. 2A). Among varied concentrations of rfhSP-D tested, a strong and maximum binding of rfhSP-D with SARS-CoV-2 S1 (5 μg/ml ) was observed at 10 μg/ml. The binding of SARS-CoV-2 to its cellular receptor, ACE2, is mediated by the RBD region of the S protein. A higher binding affinity has been reported for RBD of SARS-CoV-2 to ACE2 receptor compared to SARS-CoV. Furthermore, RBD of SARS-CoV-2 has been suggested to have a crucial role in spike protein-induced viral attachment, fusion, and entry into the host cells. In this context, this study was also aimed at determining the ability of rfhSP-D/hFL-SP-D to bind RBD of SARS-CoV-2 (FIG. 1B) via direct ELISA (RBD coated, incubated with two-fold dilutions of rfhSP-D (100 μg/ml ), and probed with anti-SP-D antibody; R&D Systems). rfhSP-D bound RBD in a dose-dependent manner. It reduced the binding affinity by maltose, but chelation of Ca²⁺ by EDTA did not significantly affect the interaction between rfhSP-D and RBD region (FIG. 2B). No rfhSP-D binding was observed in the absence of RBD, indicating a lack of non-specific interaction in this assay. To further evaluate the dose response of Maltose and EDTA (5, 10 and 20 mM), rfhSP-D was coated and probed with Si and RBD (at 2.5 and 5 μg/ml) (FIG. 2B). These results suggest that the protein-protein interaction may occur between the CRD region of rfhSP-D and the RBD region of S protein in a calcium-independent manner.

### 2. rfhSP-D Inhibits Interaction of SARS-CoV-2 S1 With Membrane Expressed ACE2 on HEK293T Cells

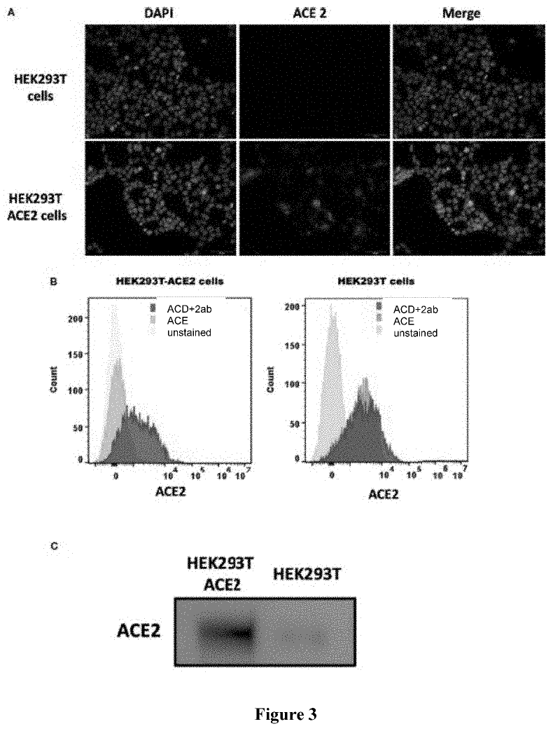

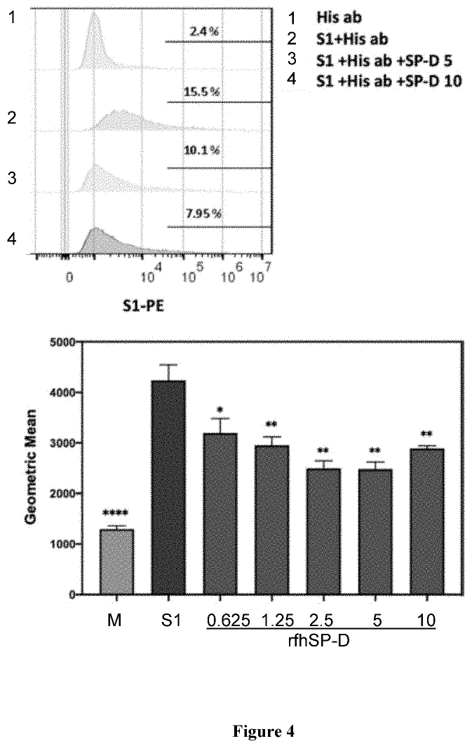

The S1 spike protein of the SARS-CoV-2 contains RBD that can recognise and interact with its cellular receptor, angiotensin converting enzyme 2 (ACE2), thus mediating viral entry into the host cells. Since rfhSP-D was found to interact with the spike protein and its RBD at the protein level, we also tested the ability of rfhSP-D to interact with HEK293T cells overexpressing ACE2 receptor. Successful transfection of the ACE2 receptor gene into HEK293T cells was verified by measuring the expression levels of ACE2 receptor via immunofluorescence microscopy (FIG. 3A), flow cytometry (FIG. 3B) and western blotting (FIG. 3C). Quantitative and qualitative analysis of the ACE2 receptor using ACE2 antibody (SN0754) revealed a higher signal for ACE2 on HEK293T-ACE2 cells when compared to HEK293T cells alone (FIGS. 3A, B). This study also focused on examining whether rfhSP-D treatment can inhibit the interaction between SARS-CoV-2 S1 and ACE2 receptor on HEK293T cells (FIG. 4). Pre-incubation of SARS-CoV-2 S1 protein (2 μg/ml ) with a varied concentration of rfhSP-D (0.625-10 μg/ml ) was found to reduce S1 binding to HEK293T cells overexpressing ACE2 receptor in a dose-dependent manner (FIG. 4). The rfhSP-D at 10 μg/ml was found to reduce the binding of S1 to ACE2 receptor on HEK293T cells by approximately 7.95% when compared to the control (S1+0 μg/ml rfhSP-D) (FIG. 4).

### 3. rfhSP-D Acts as an Entry Inhibitor of SARS-CoV-2 Infection

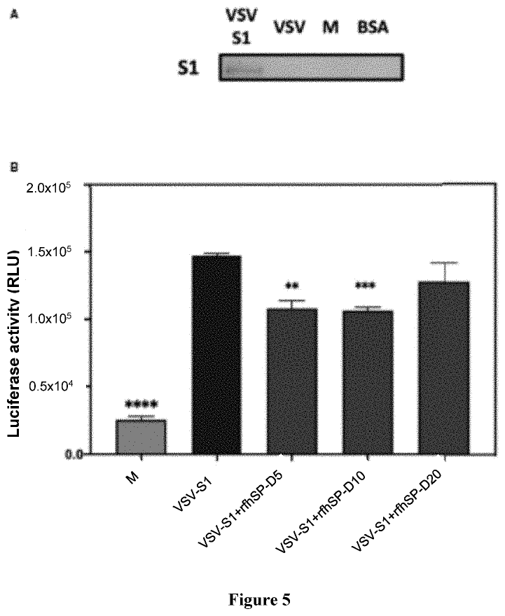

After confirming the ability of rfhSP-D to prevent the interaction between SARS-CoV-2 S1 protein and HEK293T cells overexpressing ACE2 receptor, we investigated whether rfhSP-D modulated viral entry using a luciferase reporter assay with pseudotyped lentiviral particles expressing SARS-CoV-2 S1 protein (FIG. 5). SARS-CoV-2 pseudotyped lentiviral particles were produced as a safe strategy to study the involvement of Si glycoprotein in the recognition and neutralization of the virus by a varied concentration of rfhSP-D. The production of lentiviral particles pseudotyped with envelope protein Si was carried out by co-transfecting HEK293T cells with plasmid containing the coding sequence of the indicated pcDNA3.1-nCoV-SD18 (SARS-CoV-2 spike gene), pLAS2w.Fluc.Ppuro, and pCMV-DR8.91. Purified pseudotyped particles and cell lysate harvested at 48 and 72 h were analyzed via western blotting, and the expression level of SARS-CoV-2 spike protein was determined using anti-SARS-CoV-2 (COVID-19) Spike polyclonal antibody (FIG. 5A). Cells pre-incubated rfhSP-D (5 and 10 μg/ml ) showed a significant ˜0.5 RLU fold reduction in luciferase activity (1.0×10⁵ RLU) compared to the cells+SARS-CoV-2 (1.5×10⁵ RLU) (FIG. 5B). The reduced luciferase activity, following treatment with rfhSP-D, indicated that the interaction between rfhSP-D and SARS-CoV-2 S1 protein interfered with S1-containing viral particle binding to ACE2, and hence, prevented the entry of the virus into the HEK323T-ACE2 cells (FIG. 5).

### 4. Conclusion

In this invention, the ability of the peptide rfhSP-D was confirmed to act as an entry inhibitor of pseudotyped lentiviral particles expressing SARS-CoV-2 S1 protein in hACE-2 overexpressing HEK293T cells mimicking the human SARS-CoV-2 infection. It was found in the invention that the peptide rfgSP-D could interact with the S protein of SARS-CoV, leading to an enhanced phagocytosis. It Given the findings in the invention that the peptide rfhSP-D acts as an entry inhibitor of SARS-CoV-2 infection, and is a potent innate immune molecule present in the lung surfactant, the peptide rfhSP-D is expected to play an important protective role in the pathogenesis of COVID-19.

It was confirmed in the invention that tge affinity purified, and LPS-free rfhSP-D interacted with S1 protein of SARS-CoV-2 and its receptor binding domain (RBD) in a dose-dependent manner akin to the recombinant hFL-SP-D. It was suggested in the example of inhibition of rfhSP-D binding to S protein by EDTA or maltose that the rfhSP-D bound to the carbohydrate moieties on S protein of SARS-CoV-2. It wasalso examined whether the rfhSP-D treatment can inhibit the interaction of SARS-CoV-2 S1 with ACE2 receptor on HEK293T cells. The SARSCoV-2 S1 protein (5 μg/ml ), pre-incubated with a varied concentration of rfhSP-D (0.625-10 μg/ml ), showed reduced binding to HEK293T cells overexpressing ACE2 receptor in a dose-dependent manner.

Targeting viral entry into a host cell is an emerging approach for designing and developing anti-viral therapies as viral propagation can be either restricted or blocked at an early stage of viral cycle, diminishing drug resistance by released viral particles. In the invention, the entry inhibitor role of rfhSP-D against SARS-CoV-2 was examined by luciferase reporter assay. Pseudotyped lentiviral particles were generated as a safe alternative method to mimic the structural surface of SARSCoV-2, and to test whether rfhSP-D treatment can promote or prevent viral entry into the host cells. Approximately 0.5 RLU fold reduction was seen with rfhSP-D (5 or 10 μg/ml ) treatment when compared to untreated sample (1 RLU fold; Cells+SARSCoV-2). A significantly reduced luminescent signal following rfhSP-D treatment indicated that the interaction of rfhSP-D with SARS-CoV-2-S1 restricted the binding and entry of the virus, suggestive of an entry inhibitory role of rfhSP-D against SARSCoV-2 infection.

SARS-CoV-2 mediated lung injury is correlated with diffuse alveolar damage and air space oedema, thus, accompanied by interstitial infiltration of inflammatory cells, triggering of coagulation, and fibrin deposition. Potential biomarkers to be considered during SARS infection include increased levels of inflammatory plasma makers, coagulation, and fibrinolysis. Damage to the alveolar epithelial barrier is a characteristic feature of an acute respiratory distress syndrome (ARDS) and acute lung injury (ALI); levels of plasma surfactant proteins such as SP-A and SP-D may have a prognostic value. Thus, this study prompts further investigation into the role of pulmonary surfactant in COVID-19.

In summary, the peptide rfhSP-D, containing homotrimeric neck and CRD regions, acts as an entry inhibitor of SARS-CoV-2 infection by restricting the viral entry intoHEK293T cells overexpressing ACE2 receptor. Time is ripe for taking the knowledge about the involvement of rfhSP-D and its associated anti-viral effects forward to develop a novel therapeutic approach to target multiple cellular signaling pathways. The mechanisms which enable rfhSP-D to trigger anti-viral effect are virus-specific due to the differential effect and variation in terms of the cell types and putative receptors. There is a clear therapeutic potential of rfhSP-D against SARS-CoV-2.

All publications, patents, and patent documents cited herein above are incorporated by reference herein, as though individually incorporated by reference.

The invention has been described with reference to various specific and preferred embodiments and techniques. However, one skilled in the art will understand that many variations and modifications may be made while remaining within the spirit and scope of the invention.

What is claimed is:

1. A method for treating an infection of severe acute respiratory syndrome coronavirus 2 (SARS-CoV-2) in a subject, comprising administering to said subject a therapeutically effective amount of a peptide, which is a recombinant fragment of human surfactant protein D (rfhSP-D).

2. The method of claim 1, wherein the peptide is administered in an amount effective to inhibit the entry of SARS-CoV-2 into a host cell in said subject.

3. The method of claim 1, wherein the peptide comprises a sequence as set forth in SEQ ID NO: 3

4. The method of claim 1, wherein the peptide consists of the sequence as set forth in SEQ ID NO: 3.

5. A pharmaceutical composition for treating SARS-CoV-2 infection, comprising a therapeutically effective amount of a peptide having a sequence as set forth in SEQ ID NO: 3, and a pharmaceutically acceptable carrier.

6. The pharmaceutical composition of claim 4, wherein the therapeutically effective amount is an amount effective to inhibit the entry of SARS-CoV-2 into a host cell in a subject.

7. The pharmaceutical composition of claim 4, wherein the peptide consists of the sequence as set forth in SEQ ID NO: 3.


SEQUENCE LISTING (3 sequences)

SEQ ID NO: 1 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 2 - DNA, 522 bp, Homo sapiens
ggatccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtc

SEQ ID NO: 3 - protein, 177 aa, Homo sapiens
GSPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF